(19) United States
(12) Patent Application Publication
(10) Pub. No.: US 2009/0112141 A1
(43) Pub. Date: Apr. 30, 2009

(54) METHOD AND APPARATUS FOR PROVIDING A MEDICAL DRESSING

(76) Inventor: Michael J. Derr, Quakertown, PA (US)

Correspondence Address: David Chen;Delio and Peterson LLC, 121 Whitney Avenue, New Haven, CT, 06510-1214, US

(21) Appl. No.: 11/931,766

(22) Filed: Oct. 31, 2007

(51) Int. Cl. A61F 13/00

(52) U.S. Cl. 602/53; 602/43; 602/48

(57) ABSTRACT

The invention relates to a medical dressing having an elastic material for encircling about a body member, an elastomer attached to the elastic material and adapted to directly contact a wound on the body member, and where the elastomer includes an additive for treating the wound, wherein the elastic material applies compressive force to the elastomer and wound as the elastic material encircles the body member.

### FIELD OF THE INVENTION

The invention relates to a compressive wrap with a synthetic dressing.

### BACKGROUND OF THE INVENTION

Bodily injuries, particularly those which are commonly referred to under the broad designation of contusions, wounds or burns can be painful and cause substantial discomfort to an injured person due to any tissue damage or resulting hemorrhaging of blood beneath the skin, which in turn may cause I swelling. Similar conditions can be caused by systemic diseases such as arthritis, diabetes or poor circulation. Swelling tends to make the injury more painful and disabling. It has long been recognized that, if the swelling can be minimized, then the pain and discomfort can be minimized or eliminated. Therefore, conventional practice often entails applying medicine and/or compressive force to the injured portion of the body as soon as possible after the injury in an attempt to minimize swelling. In addition, antimicrobial additives may be applied to reduce bacteria.

Compression bandages are used in particular when there is an injury to muscle fibers. Additionally compression in the form of a compression bandage has been shown to be an aid in healing of wounds and burns. In the treatment of such injuries, it has been shown that bandages, especially bandages fitted in order to prevent bruising, which exert exclusively radially inwardly directed pressure do not lead to optimal results.

However, compression bandages alone without an absorbent gauze may not effectively deal with wound exudates and are ineffective at retention of applied additives, for example antibiotics, used to treating the injury. Therefore, gauzes and the like are normally placed directly on the wound and the compressive bandage is wrapped about the gauzes.

A possible disadvantage of gauze is its limited use due to the inability of gauze to easily dissipate moisture, thereby resulting in a likely saturated gauze. At this point, the gauze is usually replaced. When ointment is added to the gauze, the wetness of the ointment may exacerbate the problem and cause the gauze to prematurely fail due to the moisture involved and where gauze, as explained above, does not efficiently absorb or dissipate moisture. Occlusive dressings are ones which seal the wound from exposure to exogenous bacteria. Gauze dressings are non-occlusive and, when wet, often serve as an ideal environment for bacterial growth.

Moreover, gauze may stick to the wound, which may also further aggravate the injury, and cause discomfort to the user when being removed. Due to the inability to efficiently dissipate moisture, gauze may not be used for prolonged periods of time, particularly when applying or reapplying ointment.

What is desired, therefore, is a bandage that continues to deliver medicine over an extended period of time. Another desire is a bandage with enhanced cushioning to protect the wound. A further desire is a bandage that provides compressive relief while delivering medicine over an extended period of time and with a reduction in failure rate in the bandage. Yet another desire is where the compression bandage is reusable by replacing the dressing or drug delivery portion of the composite. Another desire is a bandage that permits medicine to be repeatedly applied over long periods of time. Still another desire is a bandage having the ability to maintain a moist environment to aid in wound healing.

### SUMMARY OF THE INVENTION

These and other objects of the invention are achieved by a bandage that incorporates moist wound healing theories and/or the ability to deliver active ingredients including pharmaceuticals into a compression bandage structure.

Another object is a bandage that permits ointments and other medications to be applied over time with a reduction in saturation.

A further object is a bandage that applies the above advantages in addition to providing compressive relief.

Yet another object is a bandage that maintains a moist wound environment.

These and other objects are achieved by a medical dressing having an elastic material for encircling about a body member, an elastomer attached to the elastic material and adapted to directly contact a wound on the body member, and where the elastomer includes an additive for treating the contusion, wound, burn or disease wherein the elastic material applies compressive force to the elastomer and the area to be treated as the elastic material encircles the body member.

In some embodiments, the additive is a compound selected from the group consisting of an antimicrobial agent, antibacterial agent, antifungal agent, moisturizer, bactericide, anti inflammatory agent, anti allergic agent, moisturizer, menthol, analgesic, anesthetic, and combinations thereof.

In other embodiments, the elastic material is selected from the group consisting of a compression bandage, a cohesive wrap, and combinations thereof.

For either of the above embodiments, the elastomer is selected from the group consisting of a hydrophilic foam, hydrogel, hydrophobic foam, polyurethane, polyethylene, polyvinyl, alcohol, polyvinylpyrrolidone and polyethylene oxide, and combinations thereof.

In another aspect, the medical dressing includes a bandage, a hydrophilic polyurethane foam matrix attached to the bandage, and where the hydrophilic polyurethane foam matrix having at least one hydrocolloid absorptive agent dispersed throughout the hydrophilic polyurethane foam matrix.

In some embodiments, the hydrophilic polyurethane foam matrix is preformed. In other embodiments, the at least one hydrocolloid absorptive agent is selected from the group consisting of sodium carboxementylcellulose, gums, alginates, pectins, collagens, gelatins, and combinations thereof. Optionally, the at least one hydrocolloid absorptive agent is selected from the group consisting of sodium carboxementylcellulose, sodium alginate, calcium alginate, guar gum, locust bean gum, karaya gum, and combinations thereof.

In further embodiments, the preformed hydrophilic polyurethane foam layer is a polymerized composition of an aqueous mixture including water which is present in an amount from about 73% to about 99.6% by weight of the aqueous mixture and at least one hydrocolloid absorptive agent present in an amount of about 4% to about 26% by weight of the aqueous mixture; a hydrophilic polyurethane prepolymer of from about 25% to about 50% by weight of the total composition; and where the preformed matrix of the hydrophilic polyurethane foam layer has the at least one hydrocolloid absorptive agent affixed and locked into the matrix to improve the absorptive capacity of the formed hydrophilic polyurethane foam layer.

In another aspect of the invention, a method for providing a medical dressing having at least one hydrophilic polyurethane composite foam layer comprises the steps of providing a bandage; forming an aqueous mixture having at least one hydrocolloid absorptive agent and water in a quantity sufficient for the mixture; combining a hydrophilic urethane prepolymer with the aqueous mixture in a predetermined ratio to provide a polymerizing mixture for forming the matrix of the at least one layer of hydrophilic polyurethane composite form, in which the hydrocolloid absorptive agent is integrally affixed and locked into the matrix and dispersed therethrough; sizing the at least one layer of hydrophilic polyurethane composite foam; converting the foam layer into a desired shape for use; and attaching the foam layer to the bandage.

In some of these embodiments, the method for providing the medical dressing further includes adding at least one additive to the aqueous mixture and selecting the at least one additive selected from the group consisting of medicaments, proteins, enzymes, nucleic acids, soaps, hemostatic agents, antibacterial, anesthetic, antifungal, odor management agents, disinfecting and sterilizing agents, and combinations thereof.

In further embodiments, the method for providing the medical dressing includes selecting the hydrocolloid absorptive agent from the group consisting of sodium carboxementylcellulose gums, alginates, pectins, collagens, gelatins, and combinations thereof.

In yet other embodiments, providing the medical dressing includes selecting the hydrocolloid absorption agent from the group consisting of sodium carboxementylcellulose gums, alginates, pectins, collagens and gelatins; and selecting the at least one additive of the aqueous mixture from the group consisting of medicaments, proteins, enzymes, nucleic acids, soaps, hemostatic agents, antibacterial, anesthetic antifungal, disinfecting and sterilizing agents.

In another aspect of the invention, the medical dressing has a preformed hydrophilic polyurethane foam matrix having at least one hydrocolloid absorptive agent and at least one additive from the group consisting of medicaments, proteins, enzymes, nucleic acids, soaps, hemostatic agents, antibacterial, antifungal, disinfecting and sterilizing agents. The at least one additive is integrally affixed and dispersed throughout the hydrophilic polyurethane foam matrix. The polyurethane foam matrix is then attached to a bandage.

In some embodiments of this aspect, the hydrophilic polyurethane foam matrix includes an aqueous mixture including water which is present in an amount from about 15 to about 95% by weight of the aqueous mixture and at least one skin conditioning agent present in an amount from about 0.5 to about 3.5% by weight of the aqueous mixture; and a hydrophilic polyurethane prepolymer of from about 20 to about 50% by weight of the total composition.

In other embodiments of this aspect, the aqueous mixture further includes at least one surfactant present in an amount of from about 0.5 to about 5% by weight of the aqueous mixture. In further embodiments, the skin conditioning agent is selected from the group consisting of vitamins, mineral salts, trace elements, plant extracts, animal extracts, proteins, enzymes, vitamin E, vitamin A, aloe, and combinations thereof.

In yet other embodiments, the aqueous mixture further includes one or more additives selected from the group consisting of soaps, bactericides, fungicides, and combinations thereof.

The preferred embodiment of this invention is to incorporate the dressing or drug delivery matrix into the compression bandage as said dressing or drug delivery matrix is being manufactured. Optionally, any of the above embodiments, of the medical dressing or drug delivery matric can use clips, fasteners, or adhesive for holding the dressing or drug delivery matrix in place on the compression bandage. In another optional embodiment, the bacteriostatic agent is silver.

In another aspect, a method for providing a medical dressing includes the steps of providing a bandage; providing a foam pad having one or more skin conditioning agents by forming an aqueous mixture having at least one skin conditioning agent and water in a quantity sufficient for the mixture; metering a predetermined amount of hydrophilic urethane prepolymer with the aqueous mixture to form a foam layer of composite material; and converting the foam layer into a desired shape for use; and attaching the foam pad to the bandage.

In some embodiments, providing the foam pad includes the steps of metering and mixing an aqueous mixture having at least one skin conditioning agent and adequate water, with a predetermined ratio of hydrophilic urethane prepolymer to provide a polymerizing mixture for forming the foam layer of the composite material; depositing the polymerizing mixture on releasable bottom paper disposed on a moveable carrier and covering the upper surface of the polymerizing mixture with releasable top paper as the polymerizing mixture is moved with the carrier; advancing the polymerizing mixture in the top and bottom release paper by moving the carrier and sizing the foam layer being formed to the desired thickness until it is tack free; and sequentially removing the top and bottom releasable paper and simultaneously drying the sized and formed foam layer to remove residual moisture.

### BRIEF DESCRIPTION OF THE DRAWINGS

FIG. 1 depicts the medical dressing in accordance with the invention.

FIG. 2 depicts a cross section of the medical dressing shown in FIG. 1.

FIG. 3 depicts an application of the medical dressing shown in FIG. 1.

FIG. 4 depicts an embodiment of the medical dressing shown in FIG. 1.

FIG. 5 depicts a perspective view of the foam cover layer of the medical dressing shown in FIG. 1.

FIG. 6 depicts an enlarged cross-section of the foam layer of the medical dressing shown in FIG. 1.

FIG. 7 depicts a perspective view of the foam cover layer shown in FIG. 1 with a film backing.

### DETAILED DESCRIPTION

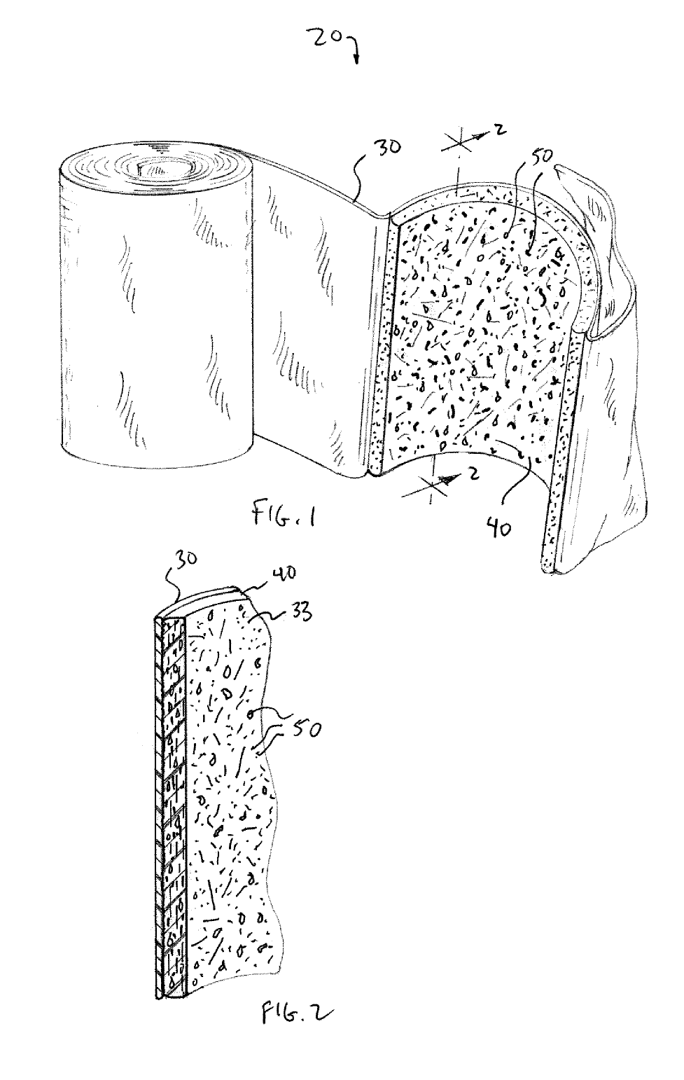

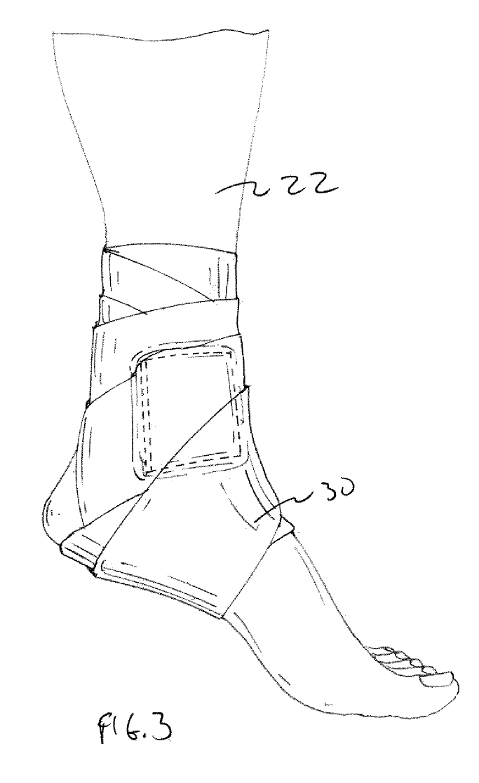

FIG. 1 depicts medical dressing 20 in accordance with the invention. Medical dressing 20 includes bandage 30 and elastomer 40. In some embodiments, additive 50 is embedded or dispersed into elastomer 40. In other embodiments, elastomer 40 absorbs additive 50. In yet other embodiments, additive 50 is adhered to elastomer 40. In further embodiments, additive 50 is mixed into elastomer 40 during formation or making of elastomer 40, which is usually in a liquid state. The finished state for elastomer 40 is cured to a solid state. In further embodiments, elastomer 40 acts as a vehicle for carrying or delivering additive 50 to body member 22 or wound, where body member 22 is any part of a wearer's body that bandage 30 may attach (see FIG. 3). Additive 50 is more particularly described below.

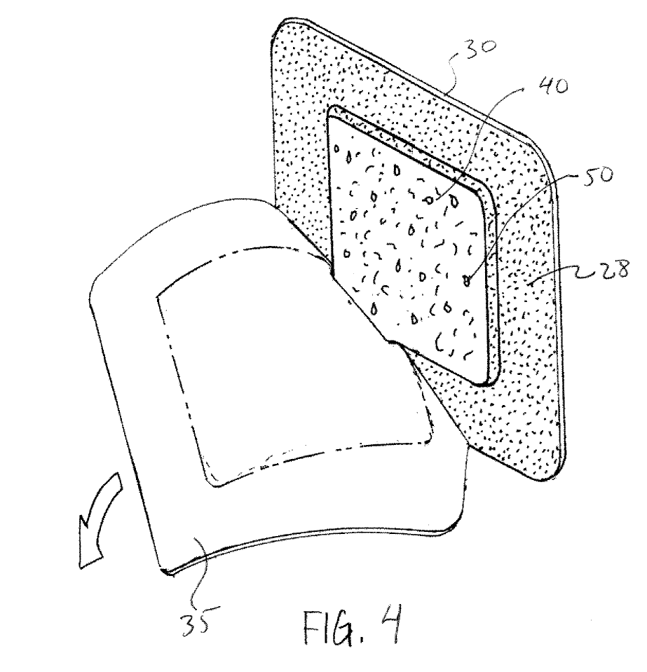

As shown, bandage 30 is an elastic material that may be wrapped or encircled about body member 22, where bandage 30 is secured by first end 32 being attached to second end 36 or middle section 34 of bandage 30 via adhesive or fasteners, such as clips that dig into the material of bandage 30. In other embodiments, bandage 30 is a compression or cohesive bandage which adheres to itself. In further embodiments, bandage 30 is a non-elastic material that is attached via adhesive to body member 22 (see FIG. 4) once backing layer 35 is removed.

Elastomer 40 is a synthetic dressing that facilitates delivery of additive 50 because elastomer 40 provides generally uniform and compressive contact to wound 51 on body member 22. By being in such contact with the wound, additive 50 within elastomer 40 is delivered to the wound in a more efficient manner than traditional bandages. Also, continuous delivery of additive 50 is achieved for certain types of additives that are embedded throughout and within elastomer 40, particularly when moisture is transferred from the wound to elastomer 40 where the moisture from wound 51 dissolves or otherwise enhances transition of additives embedded in elastomer 40 toward surface 33 of elastomer 30 and ultimately delivered to the wound. In addition, elastomer 40 molds to the shape of body member 22 and this also facilitates uniform contact with the wound.

As described above, by being in more uniform contact with the wound than traditional bandages, elastomer 40 better absorbs moisture excretions from the wound, such as blood or pus. However, elastomer 40 is better suited for absorbing moisture without failing than traditional bandages and can last longer in moist environments than natural or fibrous materials. Another benefit of elastomer 40 is that it also provides cushioning to the wound, and thereby acts as a protector from bumps or abrasions that could inhibit healing. Natural or fibrous materials tend to fully desiccate a wound site until the absorptive capacity of the material is reached, after which time excess exudate from the wound may be held beneath the dressing which may result in maceration of the surrounding tissue.

Elastomer 40 is comprised of a hydrocolloid or hydrogel absorptive agent which is dispersed into and integrally affixed to a hydrophilic polyurethane foam matrix. The hydrophilic nature of the polyurethane foam component of elastomer 40 results from the high oxyethylene content of the precursor polyol components used in its preparation. The hydrophilic foam allows the absorption of exudate from the wound without excessive desiccation of the wound site. Some of the moisture absorbed from the wound by the hydrophilic polyurethane polymer matrix may diffuse to the hydrocolloid or hydrogel absorptive agent dispersed through the foam where it may take up additives (i.e. antibiotics) held within the hydrocolloid or hydrogel and return them to the wound bed. In addition, unlike natural or fibrous materials, elastomer 40 forms an occlusive dressing, one that seals the wound and protects it from infection. The dressing formed by elastomer 40 is non-adherent and can be removed without trauma to the wound bed.

Elastomer 40 is applied as a gel, foam, or film. In some embodiments, elastomer 40 is hydrophilic. In other embodiments, elastomer 40 is hydrophobic. In use with bandage 30, elastomer 40 continuously absorbs moisture and moves it away from the wound toward the outside of elastomer 40.

Preparation of medical dressing 20 includes a hydrophilic polyurethane foam pad attached to an end of bandage 30 as described below. Those skilled in the art will recognize that the materials and reaction conditions may be varied and additional steps employed to produce hydrophilic polyurethane foams encompassed by the present invention, as demonstrated by the following examples.

An aqueous mixture of a hydrophilic polyurethane prepolymer, comprising at least one isocyanate-capped polyol, is mixed with an aqueous suspension or solution of an additive, such as a hydrocolloid absorptive agent in a predetermined ratio so that the polymerization of the polyurethane foam forms a matrix binder for the hydrocolloid absorptive agent.

The predetermined ratio is defined by the chemical nature, composition and properties which are desired to be present in the resulting polyurethane.

In another embodiment, an aqueous mixture of a hydrophilic polyurethane prepolymer, comprising at least one isocyanate-capped polyol, is mixed with a hydrogel in a predetermined ratio so that the polymerization of the polyurethane foam forms a matrix binder for the hydrogel The predetermined ratio is defined by the chemical nature, composition and properties which are desired to be present in the resulting polyurethane.

Prior to setting, the resulting prepolymerization mixture is cast as sheets or rolls or poured into molds to obtain the desired shapes of the finished foam pad. In another embodiment, the resulting mixture is cut to the desired shape after setting or curing.

The polymerization reaction time is varied from approximately 1 minute to approximately 72 hours. In a preferred embodiment, the reaction time is between approximately 5 and approximately 1200 minutes. In a more preferred embodiment, the reaction time is between approximately 15 and approximately 360 minutes. In a most preferred embodiment, the reaction time is between approximately 30 and approximately 240 minutes. In a further preferred embodiment, the reaction time is between approximately 1 and approximately 10 minutes.

Reaction temperature is varied between approximately 0° C. and approximately 100° C. In a preferred embodiment, the reaction temperature is between approximately 0 and approximately 70° C. In a more preferred embodiment, the reaction temperature is between approximately 10 and approximately 50° C. In a most preferred embodiment, the reaction temperature is between approximately 10 and approximately 45° C.

Reaction pressure is varied between approximately 0.1 atmospheres to approximately 10.0 atmospheres. In a preferred embodiment, the reaction pressure is between approximately 0.5 and approximately 5.0 atmospheres. In a more preferred embodiment, the reaction pressure is between approximately 0.5 and approximately 2.0 atmospheres. In a most preferred embodiment, the reaction pressure is between approximately 0.9 and approximately 1.1 atmospheres.

The finished or resulting hydrophilic polyurethane foam pad 40 is attached to a distal end of bandage 30 using an adhesive. In some embodiments, the prepolymerization mixture is allowed to polymerize while in direct contact with bandage 30, which allows the hydrophilic polyurethane foam pad to self-adhere to bandage 30.

The prepolymer of the instant invention is comprised of at least one isocyanate-capped polyol having a reaction functionality of at least two, the total of said polyol present having an oxyethylene content of at least 40 weight percent prior to capping.

Examples of such polyols include linear polyols formed by the reaction of ethylene oxide initiated by ethylene glycol. In some embodiments, ethylene oxide is mixed with other alkylene oxides so long as the mole percent of ethylene oxide is at least 40%. Preferably, the mole percent of ethylene oxide in such alkylene mixtures is at least 60%. Most preferably, the mole percentage of ethylene oxide in such alkylene oxide mixtures is at least 75%. Those skilled in the art will recognize other polyfunctional initiators which would provide polyols of functionality two or more. As stated, it may be desirable to include diisocyanate crosslinkers with these systems in which case said crosslinker may be included into the water into which the prepolymer is dispersed. Where the linear polyethers are mixtures of ethylene oxide with other alkylene oxides, e.g. propylene oxide, the polymer can be either random or a block copolymer and the terminal units can be either hydroxyethyl, or 1 or 2 hydroxypropyl units.

Alternatively, the class of polyols includes those with a reactive functionality of three or more. Such polyols are commonly formed by reacting alkylene oxides with a polyfunctional initiator such as trimethylolpropane or pentaerythritol. Other embodiments include polyfunctional initiators which would provide polyols of functionality three or more. Polyols of reactive functionality of three or more are formed using alkylene oxides such as ethylene oxide or mixtures of ethylene oxide with other alkylene oxides.

Alternatively, polyols of reactive functionality of two or greater may be formed by the methods described above in the presence of an initiator or crosslinker such as a polyisocyanate. Specific examples of such polyols are formed by reaction of a mixture of polyethylene glycol (m.w. approx 1000) with trimethylolpropane, trimethylolethane or glycerol with excess polyisocyanate to provide a prepolymer. Alternatively, the linear or branched polyols, (e.g. polyethylene glycol) can be reacted separately with excess polyisocyanate. The initiator, e.g. trimethylolpropane, can be separately reacted with excess polyisocyanate. Subsequently, the two capped materials are combined to form a prepolymer.

Polyisocyanates for preparing prepolymers include toluene-2,4-diisocyanate, toluene-2,6-diisocyanate, commercial mixtures of toluene-2,4- and 2,6-diisocyanates, cyclohexylene-1,4-diisocyanate, m-phenylene diisocyanate, 3,3′-diphenyl-4,4′-biphenylene diisocyanate, 4,4′-biphenylene diisocyanate, 1,6-hexamethylenediisocyanate, 1,5-naphthalenediisocyanate, cumene-2,4-diisocyanate, 2,4′-diisocyanatodiphenylether, 5,6-dimethyl-1,3-phenylenediisocyanate, 2,4-dimethyl-1,3-phenylenediisocyanate, 4,4′-diisocyanatodiphenylether, 9,10-anthracenediisocyanate, 2,4-diisocyanatostilbene, 1,4-anthracenediisocyanate, 2,4,6-toluenetriisocyanate, isophorone diisocyanate, p,p′p″-triphenylmethane triisocyanate, and combinations thereof.

Initiators for preparing prepolymers include propylene glycol, trimethylene glycol, 1,2-butylene glycol, 1,3-butanediol, 1,4-butanediol, 1,5-pentanediol, 1,2-hexane glycol, 1,10-decanediol, 1,2-cyclohexanediol, 2-butene-1,4-diol, 3-cyclohexene-1,1-dimethanol, 4-methyl-3-cyclohexene-1,1-dimethanol, 3-methylene-1,5-pentanediol, diethylene glycol, 1,2,6-hexanetriol, 1,1,1-trimethylolpropane, 3-(2-hydroxyethoxy)-1,2-propanediol, 3-(2-hydroxypropoxy)-1,2-propanediol, 2,4-dimethyl-2-(2-hydroxyethoxy)methylpentanediol-1,5,1,1,1-tris[(2-hydroxyethoxy)methyl]ethane, 1,1,1-tris[(2-hydroxypropoxy)methyl]propane, triethanolamine, triisopropanolamine, resorcinol, pyrogallol, phloroglucinol, hydroquinone, 4,6-di-tertiarybutyl catechol, catechol, resorcinol, and combinations thereof.

The water reactant phase is comprised of up to 100% water. A surfactant can be added to the water to modify the properties of the resulting foam such as the cell size or resilience. Surfactants include ethylene oxide/propylene oxide blends such as BASF F-68, F-88, L-62, L, 64, T-904 and T-1301. In other embodiments, surfactants such as Corgis Emulgrade N-1000 or Emulgin B-2, sodium lauryl sulfate, alpha olefin sulfates or Dow Chemical Triton X-100 are used. Those skilled in the art will recognize the choice of desired surfactant varies to provide the properties desired in the finished foam.

In another embodiment, the water reactant phase incorporates additives 50 into the finished foam. These include, but are not limited to, fillers such as feldspar, diatomaceous earth, silica, fibers, pharmaceutical agents such as analgesics, coagulants, anti-inflammatories, anesthetics, antihistamines, steroids, antimicrobials, antibacterials, antivirals, antifungal agents, soaps, other cleaning agents, vitamins, skin conditioners, health additives, and combinations thereof.

In one embodiment, additive 50 is dispersed with drugs, such as an over-the-counter drugs. The ranges covered include those allowed in FDA's over-the-counter drug monograph. Additive 50 includes menthol as a topical analgesic for the treatment of joint pain as an example: a regular strength product would be used on mild pain, a high strength product would be used on moderate pain, and a maximum strength product would be used on severe or intense pain.

The definition of mild, moderate, and severe pain is highly subjective in that each prospective drug has its own allowable levels for a given condition to be treated. A comparison with the drug monograph shows that the low end of the monograph corresponds to regular strength, middle part of the monograph corresponds to high strength, and top end of the monograph corresponds to maximum strength. See table A below.

In some embodiments, the monograph includes a step by step method for selling over-the counter drugs without having to get additional drug approvals from the FDA. Among the steps spelled out in the monograph are the allowable levels for each drug in an over-the-counter drug product. For example, for menthol, the FDA allows menthol levels from 1.5% to 16% to be sold as an over the counter topical analgesic provided all other requirements of the monograph are met. For each drug in the various different monographs there is a different level allowed. In other embodiments, the monograph includes 1.5% to 5%. In further embodiments, monograph is 16% for severe pain.

In a further embodiment, multiple additives 50 are dispersed in the foam, including approximately 1.5%-approximately 16.5% menthol (topical analgesic), approximately 2.0%-approximately 4% Lidocaine (topical anesthetic), approximately 0.5%-approximately 2% salacylic acid (acne), approximately 5%-approximately 10% benzoyl peroxide (acne), approximately 0.5-approximately 10% hydrocortisone, and combinations thereof. These percentages are weight percentages of finished product. These examples of additives, among others listed in FDA monographs, are incorporated into a foam or hydrogel.

In another embodiment, silver is used as additive 50 between approximately 1% and approximately 10% of dry weight, where silver is an antimicrobial and anti-odor ingredient. It functions by releasing ions which kill bacteria. A cost sensitive range is between approximately 1% and approximately 3% dry weight or weigh percentage to reduce bacterial growth. A preferred range is between approximately 4 and approximately 7%. A most preferred range is between approximately 85 and approximately 10%. The higher the percentage of silver, the greater the reduction of bacteria.

In a further embodiment, additive 50 is a property modifier, such as polyethylene glycol, glycerin and propylene glycol. These additives alone or in any combination increase softness and water wicking properties. Typical levels are between approximately 5% and approximately 15% of dry weight.

The choice of a modifier is determined by the goal to be achieved. For example, glycerine is highly effective in increasing the wicking (ability of the foam to transmit liquids along polymer matrix) properties of the foam and makes a softer foam. It also has antimicrobial properties. However it has a tendency to flash off at temperatures above 230 F., which limits its usefulness for foam which is dried. A range of glycerine is between approximately 5% and approximately 15%. Below approximately 5%, glycerine has little or negligible effects. Above approximately 15%, glycerine softens the foam to the point where the foam properties are severely degraded.

Polyethylene glycol is another modifier used in softening the foam. This is particularly effective when using hydrocolloids, which tend to make the foam hard and stiff. Polyethylene glycol reduces the hardness or stiffness caused by hydrocolloids, thereby offsetting the negative effects of hydrocolloids. In addition, polyethylene increases wicking and is less sensitive to heat and therefore is more useful in foam that is dried than glycerine. A range is between approximately 5% and approximately 20%. Below approximately 5%, it has little or negligible effects. Above approximately 20%, it softens the foam to the point where the foam properties are severely degraded.

Propylene glycol is another modifier used mainly to soften the foam. It actually decreases both the wicking and the hydrophilicity. Wicking is the ability of the foam to move liquid along the polymer. It allows liquid entering the foam at a point to spread throughout the entire foam mass. For example, wicking is desirable in a heavily exuding wound and not desirable in a dry wound. Hydrophilicity is an indication of an amount of water the foam will absorb of the foam. It is the most heat resistant of the three modifiers and would be used where relatively high heat is used to dry. It is also the most desired of the three modifiers if softness is more important than wicking. Softness is important when the ability of the foam to conform to an irregular surface (such as skin) is desired. It is also important when the product is used against damaged or tender skin. A range is between approximately 5% and approximately 10%. Below approximately 5%, it has little or negligible effects. Above approximately 10%, it softens the foam to the point where the foam properties are severely degraded.

Another additive which is used in addition to any of the above modifiers, analgesics, menthol, or silver, is a surfactant. In an optional embodiment, the surfactant is used in lieu of the above modifiers, analgesics, menthol, or silver.

This category of additives that are surfactants are used to modify the cell size, resilience, or strength of the foam. Typically, a surfactant is between approximately 0.5% and approximately 5%. Although most surfactants affect increase resilience, some surfactants, such as BASF's F-88, also increase cell size. The BASF F-88 at approximately 0.5% yields a smaller cell size than BASF F-88 at approximately 5.0%. Cell size is measured in pores per inch (PPI). A large cell is typically less than 30 PPI and a small cell is greater than 100 PPI. The middle is medium cell size. Cell size is affects the aesthetic property of the foam and, in the case of a hydrophilic foam, cell size affects absorbency rate, liquid retention, and liquid capacity. As cell size increases, rate (the speed at which liquid is absorbed) and capacity (the amount of liquid the foam can absorb as expressed as a multiple of dry weight) increase as well but retention (the amount of liquid the foam retains after squeezing) decreases.

The finished foam typically has a ratio of softness to strength that varies according to the customer's specifications. For example a 1:1 ratio of softness to strength indicates a high strength, firm foam. A ratio of 2:1 of softness to strength indicates a medium strength, softer foam. A ratio of 3:1 of softness to strength indicates a low strength, soft foam. As polymer increases in proportion to aqueous; strength increases and softness decreases. Strength is measured by tensil strength in pounds/sq. in. and high strength foam would have a tensil above 75 with low strength being under 3. Softness is measured by durometer with a reading over 100 being a relatively hard foam and under 30 being relatively soft.

Below are the descriptions of two embodiments of foams, namely a foam with a hydrocolloid agent and a foam with a skin conditioning agent.

### Foam Having at Least One Hydrocolloid Absorptive Agent

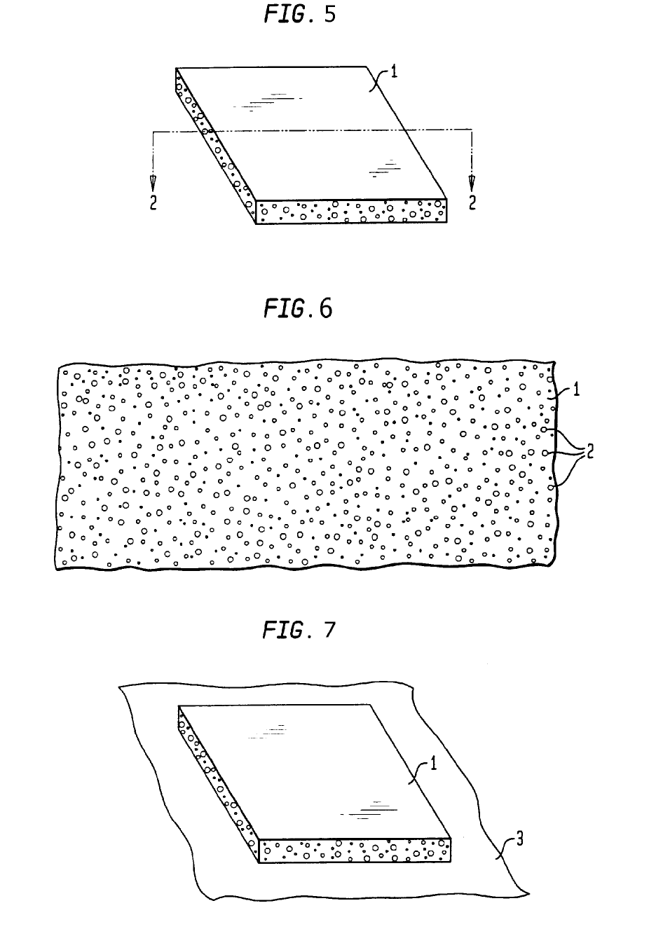

As shown in FIGS. 5, 6 and 7, the medical dressings of the present invention includes at least one monolithic, polyurethane foam layer generally designated 1 which is formed by polymerizing an aqueous mixture having one or more hydrocolloid absorptive agents with an aqueous mixture of a hydrophilic polyurethane prepolymer, in a predetermined ratio, so that the polymerization of the polyurethane foam forms a matrix binder for the one or more hydrocolloid absorptive agents. As shown in FIG. 6, the hydrocolloid absorptive agents generally designated 2 are thereby incorporated and integrally dispersed directly into the matrix of the formed foam.

The term “hydrocolloid absorptive agent” as used in accordance with the present invention includes natural, chemically modified, and synthetic hydrocolloids. Suitable hydrocolloids include, but are not limited to, natural gums such as arabic gum, ghatti gum, karaya gum, tragacanth gum, guar gum, locust bean gum and acacia gum; seaweed extracts such as agar, algin, alginate salts and carrageenin; cereal gums; starches; fermentation or microbial gums such as dextran gum and xanthan gum; pectins; gelatins; casein; and collagens. Modified forms of the hydrocolloids may also be used, including, for example, the oxidized, acetylated, carboxylated, esterified, methylated, aminated, etherated, sulfated, borated and phosphated derivatives of the hydrocolloid absorptive agents. Suitable synthetic gums include polyvinylpyrrolidone, low methoxyl pectin, propyleneglycol alginates, carboxymethyl cellulose and guar gum.

The medical dressings of the present invention are formed by metering and mixing an aqueous mixture including adequate water and at least one hydrocolloid absorptive agent with a predetermined ratio of hydrophilic urethane prepolymer to provide a prepolymerizing mixture. The hydrocolloid absorptive agents are included in the aqueous phase in an amount of about 0.5% to about 20.0% by weight. More preferably, the hydrocolloid absorptive agents are included in an amount of about 5% to about 15% by weight. Preferably, the hydrophilic urethane prepolymer is included in the aqueous phase in an amount of about 25% to about 45% by weight. Suitable surfactants, such as, for example, BASF Pluronic L 92 or BASF Pluronic F-88 (BASF, Mt. Olive, N.J.), may preferably be added to the aqueous phase. Preferably surfactants may be added in an amount of about 0.2% to about 25.0% wet weight of the aqueous phase. More preferably surfactants may be added in an amount of about 0.5% to about 2.5% wet weight of the aqueous phase.

The hydrophilic urethane prepolymer materials used in the medical dressings of the present invention are available in the commercial marketplace, and include, for example, Bipol 6, available from Mace Adhesives and Coatings, and HYPOL from Dow Chemical. General procedures for the preparation and formation of such prepolymers can be found in Polyurethanes: Chemistry and Technology by J. H. Saunders and K. C. Frisch published by John Wiley & Sons, New York, N.Y., at Vol. XVI Part 2, High Polymer Series, “Foam Systems”, pages 7-26, and “Procedures for Preparation of Polymers”, pages 26, et seq., which are hereby incorporated by reference herein.

A hydrogel is a network of polymer chains that are water soluble, sometimes found as a colloidal gel in which water is the dispersion medium. Hydrogels are superabsorbant natural or synthetic polymers. Due to their superabsorbant properties hydrogels have found use in disposable diapers, contact lenses and sanitary towels. By their aqueous nature, hydrogels are well suited and useful for the transport of soluble factors, drugs and skin conditioning agents onto the skin.

The medical and wound dressings of the present invention can also include various combinations of other ingredients without departing from the scope of the present invention, including, for example, medicaments, soaps, disinfecting and sterilizing agents, odor management agents, hemostatic agents, proteins, enzymes and nucleic acids. Preferably these agents may also be incorporated directly and dispersed throughout the prepolymerization aqueous mixture and are thereby incorporated into the foam matrix. Alternatively these other ingredients may be incorporated into the dressing by absorbing them into the formed foam cover layer following the polymerization reaction by affixing to the formed medical and wound dressings, by any suitable means, an additional layer incorporating such other ingredients, as will be understood by those skilled in the art.

Suitable medicaments, soaps, disinfecting and sterilizing agents, proteins, and enzymes are provided in the commercial market place by a myriad of suppliers and include those which aid recovery of wounds. Preferably, the medicaments include antifungal agents, antibacterial agents, angiogenesis promoting agents and the like. More preferred medicaments include antifungal agents such as metronidazole and antibacterial agents such as chlorhexidine.

Any suitable soap, disinfecting and sterilizing agent may be used. A preferred disinfecting and sterilizing agent includes hydrogen peroxide. Suitable proteins and enzymes include those which aid in wound recovery such as fibrin sealants and angiotensins, as described in U.S. Pat. Nos. 5,962,420 and 5,955,430, hereby incorporated by reference herein.

After blending and mixing the combination of the aqueous mixtures and hydrophilic urethane prepolymer, the polymerizing foam composition is preferably deposited on a releasable bottom sheet material on a movable carrier. The upper surface of the polymerizing composition is then covered with a releasable top sheet material and advanced along the moveable carrier for sizing of the foam to the desired thickness and until the foam is tack free. The releasable top and bottom sheets are then sequentially removed. When polymerization is complete, residual water may be driven off by drying the foam. Preferably, the drying is done in a drying unit at a temperature of about 200.degree. F. Drying may also be performed at lower temperatures under reduced pressures.

The medical dressings of the present invention may be formed to have any desired thickness or shape. The foam layer is preferably relatively thin. More preferably, the foam layer should have a thickness of about 1 mm to about 10 mm, and more preferably about 1.5 mm to about 6.0 mm.

Various forms of the preformed foam medical dressings of the present invention are contemplated. In a preferred embodiment, the medical dressing is an occlusive dressing which includes the foam layer and a biocompatible backing sheet or layer 3 which surrounds the foam layer. Any suitable biocompatible backing may be used, however, preferably, the backing is moisture vapor permeable. As shown in FIG. 7, in a preferred embodiment, the backing layer 3 is a hydrophilic polyurethane film which has been laminated to the foam layer 1. The backing layer 3 may extend beyond the foam layer 1 for contact with the skin of the patient.

Suitable noncytotoxic and substantially nonallergenic adhesives and/or tapes may be used to apply the medical dressings of the present invention to the skin of a patient. In a preferred embodiment, the adhesive is applied to all or a portion of the backing layer which extends beyond the foam cover layer. Preferably, the adhesives and/or tapes include those formed from polymers containing hydrophilic groups, such as hydroxyl, carboxyl, amine, amide, ether and alkoxy. More preferably, the adhesive is a pressure-sensitive acrylic adhesive. Such adhesive and tapes are well known in the art and therefore are-not more fully described.

It is to be understood that the above-detailed description of the preferred embodiment of the invention is provided by way of example only. Various details of the design, construction and composition may be modified without departing from the scope of the invention as set forth in the claims. In addition, the invention will be further described by reference to the following detailed examples. These examples are merely illustrative and not limitative of Applicants' invention in any way.

### EXAMPLE 1

An aqueous mixture comprising 4% karaya gum, water and a suitable surfactant is combined in a 60:40 ratio with hydrophilic prepolymer to form the foam.

Purified water: 190.38 parts Karaya Gum: 8.00 parts BASF Pluronic L 92: 1.60 parts BASF Pluronic F-88: 0.02 parts

Mixing was done at room temperature. L-92 and F-88 were added to water and mixed until they were completely dispersed in the water. Speed of the mixture was increased and the gum was added slowly into the vortex. Mixing was complete when the mixture was smooth (no gel particles visible). When mixing was complete, the aqueous was combined with hydrophilic prepolymer as follows

Above aqueous: 200.00 parts Hypol prepolymer: 133.33 parts.

The combination was mixed in a high speed mixing vessel until the parts were homogeneous. The combination was then poured and sized using suitable releasable substrate or substrates and allowed to cure until the foam structure was stable.

### EXAMPLE 2

An aqueous mixture comprising 2% locust bean gum, water and a suitable surfactant was combined in a 60:40 ratio with hydrophilic prepolymer to form foam.

Purified water: 194.38 parts Locust Bean Gum: 4.06 parts BASF Pluronic L 92: 1.60 parts BASF Pluronic F-88: 0.02 parts.

Mixing was done at room temperature. L-92 and F-88 were added to the water and mixed until they were completely dispersed in the water. Speed of the mixture was increased and the gum was added slowly into the vortex. Mixing was complete when the mixture was smooth (no gel particles visible). The aqueous was then combined with hydrophilic prepolymer as follows:

Above aqueous: 200.00 parts Hypol prepolymer: 100.00 parts.

The combination was mixed in a high speed mixing vessel until the parts were homogeneous. It was then poured and sized using suitable releasable substrate or substrates and allowed to cure until foam structure was stable.

### EXAMPLE 3

Comprising 4% alginate, water and a suitable surfactant was combined in a 60:40 ratio with hydrophilic prepolymer to form foam.

Purified water: 190.38 parts Kelco LVCR Alginate: 8.00 parts BASF Pluronic L 92: 1.60 parts BASF Pluronic F-88: 0.02 parts.

Mixing was done at room temperature. L-92 and F-88 were added to the water and mixed until they are completely dispersed in the water. Speed of the mixture was increased and the gum was added slowly into the vortex. Mixing was complete when the mixture was smooth (no gel particles visible). When mixing was complete, the aqueous was combined with hydrophilic prepolymer as follows:

Above aqueous: 200.00 parts Hypol prepolymer: 133.33 parts.

The combination was mixed in a high speed mixing vessel until the parts are homogeneous. It was then poured and sized using suitable releasable substrate or substrates and allowed to cure until foam structure is stable.

### EXAMPLE 4

A test was designed to measure the amount of water retained in the foam after squeezing. With the foam containing the hydrocolloid additive, a noticeable increase in the amount of retained water was observed. Results of this test using the average of five samples are as follows:

Sample Dry weight after squeezing Standard Hydrophilic foam 2.34 grams 4.53 grams (1.9× dry weight) Sample 99-309 3.39 grams 19.94 grams (5.9× dry weight) Hydrocolloid foam w. 10% alginate.

Results show that standard hydrophilic foam retains about 2. times its own weight and hydrocolloid foam retains 3 times to 8. times its own weight depending on levels of hydrocolloid used.

### Foam Having at Least One Skin Conditioning Agent

The hydrophilic foam pads of the present invention are formed by polymerizing an aqueous mixture having one or more skin conditioning agents with a predetermined quantity of a hydrophilic urethane prepolymer binder so that the polymerization of the polyurethane foam forms a matrix binder for one or more skin conditioning agents. The skin condition agents are incorporated directly into the cell structure of the foam pads and remain there until the foam pads are wetted or contacted with a sufficient moisture content. Once the pads are wetted or contacted with a sufficient moisture content, the skin conditioning agents are released from the matrix and diffuse toward the surface of the pad for contact with the skin.

The formation of the cosmetic pads in accordance with the present invention is done by metering and mixing an aqueous mixture including adequate water and at least one skin conditioning agent with a predetermined ratio of hydrophilic urethane prepolymer to provide a polymerizing mixture. The aqueous mixture includes water which is present in amounts from about 15 to about 95% by weight. The concentration of the skin conditioning agents in the aqueous phase is from about 0.5% to about 3.5%.

The skin conditioning agents of the present invention are nonvolatile agents and include vitamins, mineral salts, trace elements, plant and animal extracts, proteins, enzymes, and other agents which have therapeutic benefits for the skin. By the term “nonvolatile” it is meant that the skin conditioning agents have a high boiling or subliming temperature at normal pressures and thus do not readily evaporate at normal temperatures and pressures.

The skin conditioning agents may first be dispersed into a typical surfactant material in a prepared premix. Surfactants may be used in the aqueous solution to increase the concentration of the skin conditioning agents in the aqueous mixture. The surfactants are preferably present in amounts of about 0.5 to about 3.5% by weight of the aqueous mixture. The surfactants may be prepared from nonionic polyethylene and polypropylene oxides such as BASF surfactant available under the trademark “PLURONIC”. Other components may be added to the aqueous mixture to increase the concentration of the skin conditioning agent, such as citric acid which acts as a buffer for reducing the pH of the water component.

The aqueous mixture may further consist of various combinations of other components without departing from the scope of the present invention, including, for example, soaps, bactericides and fungicides. Bactericides are provided in the commercial marketplace by a myriad of suppliers for controlling bacterial and fungal growth. One preferred material is supplied by Lauricidin Co. of Galena, Ill. 61036, under the trademark “LAURICIDIN”.

The hydrophilic urethane prepolymer component of the present invention is available in the commercial marketplace. Suitable prepolymers will be readily recognized by those of ordinary skill in the art and are described in prior art U.S. Pat. Nos. 4,137,200; 4,209,605; 3,805,532; 2,999,013 and general procedures for the preparation and formation of such prepolymers can be found in Polyurethane's Chemistry and Technology by J. H. Suanders and K. C. Frisch published by John Wiley & Sons, New York, N.Y., at Vol. XVI Part 2, High Polymer Series, “Foam Systems”, pages 7-26, and “Procedures for the Preparation of Polymers”, pages 26 et seq. One preferred prepolymer for use in the present invention is Bipol 6 available from Mace Adhesives and Coatings. The hydrophilic urethane prepolymer is present in amount of about 20 to about 50% by weight of the total composition.

As will be appreciated by those skilled in the art, the cosmetic pads of the present invention can be formed to have any desired thickness and shape. After blending and mixing the combination of the aqueous mixtures and hydrophilic urethane prepolymer, the polymerizing foam pad composition is preferably deposited on a releasable bottom paper on a movable carrier. The upper surface of the polymerizing composition is then covered with a releasable top paper and advanced along the moveable carrier for sizing of the foam to the desired thickness and until the foam is tack free. The top and bottom releasable paper are sequentially removed. When polymerization is complete, residual water may be driven off by drying the foam in a drying unit at a temperature of about 200.degree. F.

Preferably the foam pads of the present invention have a thickness of about 1 mm to about 40 mm. The foam pad may initially be formed into large blocks which are then cut into any desired shape.

In some embodiments, the bacteriostatic agent is silver. Silver is incorporated as elemental silver or used as a compound such as silver chloride. In either case silver ions are released to kill germs.

Elemental silver has more ions to be released but it can be toxic if large amounts of free silver are available to the skin or wound bed. We have chosen to use a form of elemental silver which involves permanently bonding an ultra thin layer of the silver around a synthetic fiber core. Preferred fibers are polyester and nylon. The result is a large surface area of elemental silver which allows for a high ion release level. At the same time the binding to the fiber core prevents the release of silver to the skin or wound bed.

### EXAMPLE 5

| Ingredients | Percent by Weight |
|---|---|
| Water | 93.06% |
| Surfactant (BASF F88 Pluronic) | 1.58% |
| Burgess Clay (Wolastinite) | 4.15% |
| Bactericide | .28% |
| Vitamin E | 1.23% |

This aqueous mixture was then metered with the hydrophilic prepolymer at a ratio of 2 parts aqueous to 1 part polymer by weight and dispensed onto a moving casting liner. After polymerization, the web is sized and compressed to achieve the target thickness.

### EXAMPLE 6

| Ingredients | Percent by Weight |
|---|---|
| Water | 93.06% |
| Surfactant (BASF F88 Pluronic) | 1.58% |
| Burgess Clay (Wolastinite) | 4.15% |
| Bactericide | .28% |
| Vitamin A | 1.23% |

This aqueous mixture was then metered and mixed with a hydrophilic prepolymer in a ratio of 3.2 parts to 1 part by weight. After polymerization the web is sized and compressed to desired thickness.

### EXAMPLE 7

| Ingredients | Percent by Weight |
|---|---|
| Water | 92.11% |
| Surfactant (BASF F88 Pluronic) | 1.58% |
| Surfactant (BASF L62 Pluronic) | 1.25% |
| Burgess Clay (Wolastinite) | 4.15% |
| Bactericide | .28% |
| Aloe | .93% |

This aqueous mixture was then metered and mixed with a hydrophilic prepolymer in a ratio of 2.7 parts to 1 by weight. After polymerization the web is sized and compressed to desired thickness.

The embodiments described above should provide adequate details of the invention. However, it should be noted by those skilled in the art that the disclosures herein are exemplary only and that various other alternatives, adaptations and modifications may be made within the scope of the present invention. Accordingly, the present invention is not limited by the specific embodiments as illustrated.

What is claimed is:

1. An elastic material for encircling about a body member;
an elastomer attached to said elastic material and adapted to directly contact a wound on the body member; and
wherein said elastic material applies compressive force to said elastomer and wound as said elastic material encircles the body member.

2. The medical dressing according to claim 1, wherein said elastomer includes an additive for treating the wound or medical condition

3. The medical dressing according to claim 1, wherein said additive is a compound selected from the group consisting of an antimicrobial agent, antibacterial agent, antifungal agent, moisturizer, bactericide, anti inflammatory agent, anti allergic agent, moisturizer, menthol, analgesic, anesthetic, and combinations thereof.

4. The medical dressing according to claim 1, wherein said elastic material is selected from the group consisting of a compression bandage, a cohesive wrap, and combinations thereof.

5. The medical dressing according to claim 1, wherein said elastomer is selected from the group consisting of a hydrophilic foam, hydrogel, hydrophobic foam, polyurethane, polyethylene, polyvinyl, alcohol, polyvinylperilidone, polyethylene oxide, and combinations there

6. The medical dressing according to claim 1 wherein said additive is a pharmaceutically active compound.

7. A medical dressing, comprising:
a bandage;
a hydrophilic polyurethane foam matrix attached to said bandage; and
said hydrophilic polyurethane foam matrix having at least one hydrocolloid absorptive agent dispersed throughout said hydrophilic polyurethane foam matrix.

8. The medical dressing according to claim 7, wherein said hydrophilic polyurethane foam matrix is preformed.

9. The medical dressing of claim 7, wherein said at least one hydrocolloid absorptive agent is selected from the group consisting of gums, alginates, pectins, collagens, gelatins, sodium alginate, calcium alginate, guar gum, locust bean gum, karaya gum, sodium carboxymethylcellulose and combinations thereof.

10. The medical dressing according to claim 7, wherein said hydrophilic polyurethane foam layer comprises a preformed matrix from a polymerized composition of:
an aqueous mixture including water which is present in an amount from about 73% to about 99.6% by weight of the aqueous mixture and at least one hydrocolloid absorptive agent present in an amount of about 4% to about 26% by weight of the aqueous mixture;
a hydrophilic polyurethane prepolymer of from about 25% to about 50% by weight of the total composition; and
said preformed matrix of the hydrophilic polyurethane foam layer having said at least one hydrocolloid absorptive agent affixed and locked into the matrix to improve the absorptive capacity of the formed hydrophilic polyurethane foam layer.

11. A method for providing a medical dressing having at least one hydrophilic polyurethane composite foam layer comprising the steps of:
providing a bandage;
forming an aqueous mixture having at least one hydrocolloid absorptive agent and water in a quantity sufficient for the mixture;
combining a hydrophilic urethane prepolymer with said aqueous mixture in a predetermined ratio to provide a polymerizing mixture for forming the matrix of the said at least one layer of hydrophilic polyurethane composite form, in which the hydrocolloid absorptive agent is integrally affixed and dispersed therethrough;
sizing said at least one layer of hydrophilic polyurethane composite foam;
converting the foam layer into a desired shape for use; and
attaching the foam layer to the bandage.

12. The method for providing a medical dressing according to claim 11, further comprising the step of adding at least one additive to the aqueous mixture and selecting said at least one additive selected from the group consisting of medicaments, proteins, enzymes, nucleic acids, soaps, hemostatic agents, antibacterial, antifungal, odor management agents, disinfecting and sterilizing agents, and combinations thereof.

13. The method for providing a medical dressing according to claim 11, further comprising the step of selecting the hydrocolloid absorptive agent from the group consisting of gums, alginates, pectins, collagens, gelatins, and combinations thereof.

14. The method for providing a medical dressing as in claim 11, further comprising the step of:
selecting the hydrocolloid absorption agent from the group consisting of gums, alginates, pectins, collagens and gelatins; and
selecting the at least one additive of the aqueous mixture from the group consisting of medicaments, proteins, enzymes, nucleic acids, soaps, hemostatic agents, antibacterial, antifungal, disinfecting and sterilizing agents.

15. A medical dressing, comprising:
a preformed hydrophilic polyurethane foam matrix having at least one hydrocolloid absorptive agent and at least one additive from the group consisting of medicaments, proteins, enzymes, nucleic acids, soaps, hemostatic agents, antibacterial, antifungal, disinfecting and sterilizing agents;
said at least one additive is affixed and dispersed throughout said hydrophilic polyurethane foam matrix;
a bandage; and
said polyurethane foam matrix being attached to said bandage.

16. The medical dressing according to claim 15, wherein said hydrophilic polyurethane foam matrix includes:
an aqueous mixture including water which is present in an amount from about 15 to about 95% by weight of the aqueous mixture and at least one skin conditioning agent present in an amount from about 0.5 to about 3.5% by weight of the aqueous mixture; and
a hydrophilic polyurethane prepolymer of from about 20 to about 50% by weight of the total composition.

17. The medical dressing of claim 16, wherein the aqueous mixture further includes at least one surfactant present in an amount of from about 0.5 to about 5% by weight of the aqueous mixture.

18. The medical dressing of claim 16, wherein the skin conditioning agent is selected from the group consisting of vitamins, mineral salts, trace elements, plant extracts, animal extracts, proteins, enzymes, vitamin E, vitamin A, aloe, and combinations thereof.

19. The medical dressing of claim 16, wherein the aqueous mixture further includes one or more additives selected from the group consisting of soaps, bactericides, fungicides, and combinations thereof.

20. The medical dressing according to claim 16, further comprising clips for holding the bandage in place.

21. The medical dressing according to claim 16, wherein said bacteriostatic agent is silver.

22. A method for providing a medical dressing comprising the steps of:
providing a bandage;
providing a foam pad having one or more skin conditioning agents by forming an aqueous mixture having at least one skin conditioning agent and water in a quantity sufficient for the mixture;
metering a predetermined amount of hydrophilic urethane prepolymer with said aqueous mixture to form a foam layer of composite material; and
converting the foam layer into a desired shape for use; and attaching the foam pad to the bandage.

23. The method according to claim 22, wherein providing a foam pad includes the steps of:
metering and mixing an aqueous mixture having at least one skin conditioning agent and adequate water, with a predetermined ratio of hydrophilic urethane prepolymer to provide a polymerizing mixture for forming the foam layer of the composite material;
depositing the polymerizing mixture on releasable bottom paper disposed on a moveable carrier and covering the upper surface of the polymerizing mixture with releasable top paper as the polymerizing mixture is moved with the carrier;
advancing the polymerizing mixture in the top and bottom release paper by moving the carrier and sizing the foam layer being formed to the desired thickness until it is tack free; and
sequentially removing the top and bottom releasable paper and simultaneously drying the sized and formed foam layer to remove residual moisture.

24. A medical dressing comprising:
A hydrogel constructed of:
to 90 percent by weight of water
A polymer in the amount of 1 percent to 35 percent chosen from:
polyurethane, polyethylene, polyvinyl, alcohol, polyvinylperilidone and polyethylene oxide, and combinations thereof;
Said polymer being further cross-linked either chemically or with some form of high energy such as electron beam radiation of uv light.

25. The method for providing a medical dressing according to claim 24, further comprising the step of adding at least one additive to the aqueous mixture and selecting said at least one additive selected from the group consisting of medicaments, proteins, enzymes, nucleic acids, soaps, hemostatic agents, antibacterial, antifungal, odor management agents, disinfecting and sterilizing agents, humectants and combinations thereof.
